(19) United States
(12) Patent Application Publication
(10) Pub. No.: US 2006/0217360 A1
(43) Pub. Date: Sep. 28, 2006

(54) Tubulysin biosynthesis gene

(76) Inventors: Gerhard Hoefle, Braunschweig (DE); Rolf Mueller, Braunschweig (DE); Florenz Sasse, Braunschweig (DE); Axel Sandmann, Braunschweig (DE); Helmut Bloecker, Braunschweig (DE)

Correspondence Address: Ronald R Santucci;Frommer Lawrence & Haug, 745 Fifth Avenue, New York, NY, 10151, US

(21) Appl. No.: 10/526,572

(22) PCT Filed: Sep. 3, 2003
(86) PCT No.: PCT/EP03/09780

§ 371 Date: Oct. 11, 2005

(30) Foreign Application Priority Data
Sep. 5, 2002 (DE) 102 41 152.2

(51) Int. Cl. C12Q 1/68; C07H 21/04; C12P 21/06; C12P 17/00; A61K 31/33; C12N 15/74

(52) U.S. Cl. 514/183; 435/6; 435/69.1; 435/117; 435/252.3; 435/471; 536/23.2

(57) ABSTRACT

The invention relates to an ssDNA molecule, to a dsDNA molecule, to an RNA, to a vector, to the use thereof and to a cell, especially for the expression of tubulysin genes.

Tubulysins have already been put forward, in Irsee, as a new family of substances from Myxobacteria, which acts on the tubulin skeleton; cf. PCT/EP 97/05095 and DE 100 08 089.8 and the literature cited therein. In contrast to epothilones, they exhibit a microtubule-degrading action and increased formation of centrosomes. With a cytotoxicity of IC₅₀=10-500 pg, tubulysins are especially interesting as potential cytostatic agents.

Tubulysins have a cytostatic or antimitotic action on fungi, human tumours or cancer cell lines and other animal cell cultures (cf. Table). Within the cells, they result in rapid degradation of the microtubule structure. The actin skeleton is preserved. Under the influence of tubulysins, adherently growing L929 mouse cells increase in volume without dividing and develop large cell nuclei, which then break up in an apoptotic process.

Spectrum of action

Inhibition zone [mm]

| Fungi | Tubulysin A | Tubulysin B |
|---|---|---|
| Aspergillus niger | 20 | 18 |
| Botrytis cineria | 23 | 18 |
| Coprinus cinereus | 20 | |
| Pythinum debaryanum | 20 | |

| | IC₅₀ [ng/ml] | | |
|---|---|---|---|
| | Tubulysin A | Tubulysin B | Tubulysin C |
| Human cancer cell line | | | |
| KB-3-1 | 0.01 | 0.02 | 0.1 |
| (DSM ACC 158) | | | |
| K-562 | 0.1 | 0.2 | 1.5 |
| (ATCC CCL 243) | | | |
| HL-60 | 0.04 | 0.08 | 0.4 |
| (ATCC-CCL 240) | | | |
| Animal cell lines | | | |
| L929, mouse | 0.2 | 0.4 | 2 |
| (ATCC CCL1) | | | |
| Pt K2, Potorous tridactylis | 0.2 | 0.2 | 2 |
| (ATCC CCL 56) | | | |

Agar diffusion test: 20 μg per test disc of 6 mm diameter

According to one embodiment, the invention relates to an ssDNA molecule selected from the following group:
(i) an ssDNA molecule having a sequence according to FIG. 1;
(ii) an ssDNA molecule which is 90, 91, 92, 93, 94, 95, 96, 97, 98, 99 or 100% homologous to an ssDNA molecule according to (i) in respect of its number of nucleotides or its nucleotide sequence but which differs by at least one nucleotide from the ssDNA molecule according to (i) in respect of its number of nucleotides and/or its nucleotide sequence; and
(iii) an ssDNA molecule having a sequence which is complementary to the sequence of an ssDNA molecule according to (i) or (ii).

The invention relates furthermore to a dsDNA molecule comprising an ssDNA molecule according to the invention and a strand complementary thereto.

According to a further embodiment, the invention relates to an ssDNA molecule selected from the following group:
(i) an ssDNA molecule having a sequence of positions 3,308 to 1 (ORF 16) of the sequence according to FIG. 1;
(ii) an ssDNA molecule having a sequence of positions 4706 to 3453 (ORF 15) of the sequence according to FIG. 1;
(iii) an ssDNA molecule having a sequence of positions 5719 to 7164 (ORF 14) of the sequence according to FIG. 1;
(iv) an ssDNA molecule having a sequence of positions 9557 to 7317 (ORF 13) of the sequence according to FIG. 1;
(v) an ssDNA molecule having a sequence of positions 12193 to 10550 (ORF 12) of the sequence according to FIG. 1;
(vi) an ssDNA molecule having a sequence of positions 12841 to 13881 (ORF 11) of the sequence according to FIG. 1;
(vii) an ssDNA molecule having a sequence of positions 14833 to 13835 (ORF 10) of the sequence according to FIG. 1;
(viii) an ssDNA molecule having a sequence of positions 14942 to 15586 (ORF 9) of the sequence according to FIG. 1;
(ix) an ssDNA molecule having a sequence of positions 15.847 to 16983 (ORF 8) of the sequence according to FIG. 1;
(x) an ssDNA molecule having a sequence of positions 21154 to 18809 (ORF 7) of the sequence according to FIG. 1;
(xi) an ssDNA molecule having a sequence of positions 22366 to 23532 (ORF 6) of the sequence according to FIG. 1;
(xii) an ssDNA molecule having a sequence of positions 24591 to 26513 (ORF 5) of the sequence according to FIG. 1;
(xiii) an ssDNA molecule having a sequence of positions 26597 to 27517 (ORF 4) of the sequence according to FIG. 1;
(xiv) an ssDNA molecule having a sequence of positions 29858 to 30400 (ORF 3) of the sequence according to FIG. 1;
(xv) an ssDNA molecule having a sequence of positions 31220 to 32392 (TubA) of the sequence according to FIG. 1;
(xvi) an ssDNA molecule having a sequence of positions 33056 to 32397 (ORF 2) of the sequence according to FIG. 1;
(xvii) an ssDNA molecule having a sequence of positions 34195 to 33074 (TubZ) of the sequence according to FIG. 1;
(xviii) an ssDNA molecule having a sequence of positions 35422 to 34205 (ORF 1) of the sequence according to FIG. 1;
(xix) an ssDNA molecule having a sequence of positions 35522 to 40147 (TubB) of the sequence according to FIG. 1;
(xx) an ssDNA molecule having a sequence of positions 40144 to 48021 (TubC) of the sequence according to FIG. 1;
(xxi) an ssDNA molecule having a sequence of positions 48011 to 58558 (TubD) of the sequence according to FIG. 1;
(xxii) an ssDNA molecule having a sequence of positions 58551 to 62096 (TubE) of the sequence according to FIG. 1;
(xxiii) an ssDNA molecule having a sequence of positions 62103 to 70616 (TubF) of the sequence according to FIG. 1;
(xxiv) an ssDNA molecule which is hybridisable with a molecule according to (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii) or (xxiii) under stringent conditions and especially has the same number of bases; and
(xxv) an ssDNA molecule which is 90, 91, 92, 93, 94, 95, 96, 97, 98, 99 or 100% homologous to an ssDNA molecule according to (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii) or (xxiii) in respect of its number of nucleotides or its nucleotide sequence but which differs by at least one nucleotide from that ssDNA molecule in respect of its number of nucleotides and/or its nucleotide sequence; and
(xxvi) an ssDNA molecule having a sequence which is complementary to the sequence of a molecule according to (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii), (xxiii), (xxiv) or (xxv).

The invention relates furthermore to a dsDNA molecule comprising such an ssDNA molecule according to the invention and a strand complementary thereto.

According to a further embodiment, the invention relates to an ssDNA molecule selected from the following group:
(i) an ssDNA molecule having a sequence of positions 35747 to 36769 (domain C of the tubB gene) of the sequence according to FIG. 1;
(ii) an ssDNA molecule having a sequence of positions 37184 to 39817 (domain A of the tubB gene) of the sequence according to FIG. 1;
(iii) an ssDNA molecule having a sequence of positions 38369 to 39730 (domain NMT of the tubB gene) of the sequence according to FIG. 1;
(iv) an ssDNA molecule having a sequence of positions 39818 to 40069 (domain PCP of the tubB gene) of the sequence according to FIG. 1;
(v) an ssDNA molecule having a sequence of positions 40372 to 41397 (domain C of the tubC gene) of the sequence according to FIG. 1;
(vi) an ssDNA molecule having a sequence of positions 41824 to 43215 (domain A of the tubC gene) of the sequence according to FIG. 1;
(vii) an ssDNA molecule having a sequence of positions 43216 to 43461 (domain PCP of the tubC gene) of the sequence according to FIG. 1;
(viii) an ssDNA molecule having a sequence of positions 43552 to 44574 (domain C of the tubC gene) of the sequence according to FIG. 1;
(ix) an ssDNA molecule having a sequence of positions 44980 to 47631 (domain A of the tubC gene) of the sequence according to FIG. 1;
(x) an ssDNA molecule having a sequence of positions 46153 to 47547 (domain NMT of the tubC gene) of the sequence according to FIG. 1;
(xi) an ssDNA molecule having a sequence of positions 47632 to 47868 (domain PCP of the tubC gene) of the sequence according to FIG. 1;
(xii) an ssDNA molecule having a sequence of positions 48011 to 49321 (domain KS of the tubD gene) of the sequence according to FIG. 1;
(xiii) an ssDNA molecule having a sequence of positions 49622 to 50584 (domain AT of the tubD gene) of the sequence according to FIG. 1;
(xiv) an ssDNA molecule having a sequence of positions 51473 to 52309 (domain KR of the tubD gene) of the sequence according to FIG. 1;
(xv) an ssDNA molecule having a sequence of positions 53066 to 53980 (domain ER of the tubD gene) of the sequence according to FIG. 1;
(xvi) an ssDNA molecule having a sequence of positions 54158 to 54460 (domain ACP of the tubD gene) of the sequence according to FIG. 1;
(xvii) an ssDNA molecule having a sequence of positions 54461 to 55870 (domain HC of the tubD gene) of the sequence according to FIG. 1;
(xviii) an ssDNA molecule having a sequence of positions 56000 to 57412 (domain A of the tubD gene) of the sequence according to FIG. 1;
(xix) an ssDNA molecule having a sequence of positions 57413 to 57643 (domain PCP of the tubD gene) of the sequence according to FIG. 1;
(xx) an ssDNA molecule having a sequence of positions 58689 to 59714 (domain C of the tubE gene) of the sequence according to FIG. 1;
(xxi) an ssDNA molecule having a sequence of positions 60156 to 61697 (domain A of the tubE gene) of the sequence according to FIG. 1;
(xxii) an ssDNA molecule having a sequence of positions 61698 to 61967 (domain PCP of the tubE gene) of the sequence according to FIG. 1;
(xxiii) an ssDNA molecule having a sequence of positions 62127 to 63320 (domain KS of the tubF gene) of the sequence according to FIG. 1;
(xxiv) an ssDNA molecule having a sequence of positions 63711 to 64676 (domain AT of the tubF gene) of the sequence according to FIG. 1;
(xxv) an ssDNA molecule having a sequence of positions 64959 to 65882 (domain KR of the tubF gene) of the sequence according to FIG. 1;
(xxvi) an ssDNA molecule having a sequence of positions 65985 to 67061 (domain CMT of the tubF gene) of the sequence according to FIG. 1;
(xxvii) an ssDNA molecule having a sequence of positions 67242 to 67829 (domain DH of the tubF gene) of the sequence according to FIG. 1;
(xxviii) an ssDNA molecule having a sequence of positions 68247 to 69128 (domain ER of the tubF gene) of the sequence according to FIG. 1;
(xxix) an ssDNA molecule having a sequence of positions 69360 to 69605 (domain PCP of the tubF gene) of the sequence according to FIG. 1;
(xxx) an ssDNA molecule having a sequence of positions 69759 to 70586 (domain TE of the tubF gene) of the sequence according to FIG. 1;
(xxxi) an ssDNA molecule which is hybridisable with a molecule according to (i), (ii), (iii), (iv), (v), (vi), (vii), (viii), (ix), (x), (xi), (xii), (xiii), (xiv), (xv), (xvi), (xvii), (xviii), (xiv), (xx), (xxi), (xxii), (xxiii), (xxiv), (xxv), (xxvi), (xxvii), (xxviii), (xxix) or (xxx) under stringent conditions and especially has the same number of bases;
(xxxii) an ssDNA molecule which is 90, 91, 92, 93, 94, 95, 96, 97, 98, 99 or 100% homologous to an ssDNA molecule according to (i), (ii), (iii), (iv), (v), (vi), (vii), (viii), (ix), (x), (xi), (xii), (xiii), (xiv), (xv), (xvi), (xvii), (xviii), (xiv), (xx), (xxi), (xxii), (xxiii), (xxiv), (xxv), (xxvi), (xxvii), (xxviii), (xxix) or (xxx) in respect of its number of nucleotides or its nucleotide sequence but which differs by at least one nucleotide from that ssDNA molecule in respect of its number of nucleotides and/or its nucleotide sequence; and
(xxxiii) an ssDNA molecule having a sequence which is complementary to the sequence of a molecule according (i), (ii), (iii), (iv), (v), (vi), (vii), (viii), (ix), (x), (xi), (xii), (xiii), (xiv), (xv), (xvi), (xvii), (xviii), (xiv), (xx), (xxi), (xxii), (xxiii), (xxiv), (xxv), (xxvi), (xxvii), (xxviii), (xxix), (xxx), (xxxi) or (xxxii).

The invention relates furthermore to a dsDNA molecule comprising such an ssDNA molecule and a strand complementary thereto.

The invention relates furthermore to variants or mutants which result from a substitution, insertion or deletion of nucleotides or from an inversion of nucleotide segments of an ssDNA molecule according to the invention or of a dsDNA molecule according to the invention, those variants and mutants encoding enzyme variants or enzyme mutants for the production of secondary substance(s) having the properties characteristic of tubulysins described at the beginning, especially having cytostatic action. The person skilled in the art will be familiar with mass screening.

The invention relates furthermore to RNA
(a) having a sequence corresponding to that of an ssDNA molecule according to the invention or
(b) having a sequence of an RNA according to (a) but in the opposite direction (anti-sense), or
(c) having a sequence of an RNA according to (a) or (b) and having a strand complementary thereto,
   in each case optionally as an element of a recombinant vector.

In accordance with a further embodiment, the invention relates to a recombinant vector, especially an expression vector, having a DNA molecule according to the invention.

In accordance with a further embodiment, the invention relates to a cell, especially for expression, into which a DNA molecule according to the invention or a vector according to the invention has been integrated.

The cell according to the invention can be derived from culturable bacteria such as Myxobacteria such as Angiococcus, especially A. disciformis, Archangium, especially A. gephyra, Escherichia coli, pseudomonads or actinomycetes.

In accordance with a further embodiment, the invention relates to use of a vector according to the invention for the transformation of cells or organisms for the transient or permanent expression of one or more proteins (expression product(s) which is/are encoded by a DNA (ssDNA or dsDNA) of the vector).

In accordance with a further embodiment, the invention relates to use of a cell according to the invention for the enzymatic biosynthesis, metasynthesis or partial synthesis of a tubulysin, especially tubulysin A, B, C, D, E and/or F.

In accordance with a further embodiment, the invention relates to an expression product of a DNA molecule according to the invention or of a vector according to the invention or of a cell according to the invention.

The present invention relates especially to a polynucleotide comprising a sequence as defined in SEQ ID NO: 1, 18, 33 or 36 or a fragment thereof. SEQ ID NO: 1 and 18 describe the (+) and (−) strands, respectively, of the tubulysin biosynthesis cluster of Angiococcus disciformis. SEQ ID NO: 33 is a sequence comprising several overlapping genes of the cluster. SEQ ID NO: 36 describes a mutant of Angiococcus disciformis. It was found, surprisingly, that this mutant exhibited tubulysin D production many times that of the wild type. The tubulysin overexpression, in terms of the overall activity of all tubulysin derivatives, is even higher that that of tubulysin D, which on no account was to be expected. The genes of SEQ ID NO: 36 are clearly involved in the negative regulation of tubulysin expression. This mutant is, by virtue of the increased expression of all tubulysins, especially suitable for the production of the polypeptides according to the invention. Antibodies against the wild type expression products of that sequence can be used to minimise their negative influence on tubulysin production even in other strains. Antisense-RNA or RNAi techniques which interact with the wild type sequence of the negative regulator genes also have a similar effect.

The fragments of the polynucleotide may have any desired partial sequence and length, but preference is given to those fragments which encode proteins. The polynucleotides of the present invention also include, but are not limited to, a polynucleotide which hybridises at the complementary strand of the disclosed nucleotide sequences under moderately stringent or stringent conditions; a polynucleotide which is an allele variant of any polynucleotide described above; a polynucleotide which encodes a species homologue of any of the proteins disclosed herein; and a polynucleotide which encodes a polypeptide which has an additional specific domain or a truncation or shortening of the disclosed proteins.

The term “CDS” denotes a sequence of nucleotides which corresponds to the sequence of amino acids in a protein, that is to say the amino-acid-encoding sequence regions, including the respective start and stop codons.

In a preferred embodiment, the polynucleotide according to the invention is a fragment which is a CDS defined in the sequence protocol.

The present invention relates furthermore to a vector comprising a polynucleotide as described above. Vectors for various purposes are known in the prior art, as well as the techniques for subcloning polynucleotides into such vectors. These are described in the new edition of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (D. N. Glover ed., 1985); Gene Transfer Vectors for Mammalian Cells (Miller & Calos, eds.); Current Protocols in Molecular Biology and Short Protocols in Molecular Biology, 3rd Edition (F. M. Ausubel et al., eds.); Recombinant DNA Methodology (R. Wu ed., Academic Press) or “A Practical Guide To Molecular Cloning”. Examples of vectors are to be found, inter alia, in Gene Transfer Vectors For Mammalian Cells (J. H. Miller and M. P. Calos eds., 1987, Cold Spring Harbor Laboratory).

The vector is preferably an expression vector, that is to say in general a plasmid, a phage, a virus or a vector for expressing a polypeptide from a DNA (RNA) sequence. An expression vector can encompass a transcription unit which has an arrangement of the following: (1) a genetic element or elements with a regulatory role in gene expression, for example promoters or enhancers, (2) a structural sequence or coding sequence which is transcribed into mRNA and translated into a protein and (3) suitable transcription initiation and termination sequences. Structural units which are provided for use in yeasts or eukaryotic expression systems preferably include a leader sequence which makes possible extracellular secretion of a translated protein by a host. Alternatively, when a recombinant protein without a leader or transport sequence is expressed, it may include an N-terminal methionine residue. That residue may, but need not, be removed from the expressed recombinant protein subsequently in order to obtain the end product.

The present invention relates furthermore to a cell comprising such a vector. The vector can be introduced into the cell by means of the known techniques such as, for example, transfection, electroporation, lipofection etc. In the case of viral vectors, infection is also possible. The cells may be eukaryotic or prokaryotic cells.

The methods for selecting and propagating the cells comprising the vector will also be known to the person skilled in the art. Examples of the culturing of cells of animal origin are to be found, inter alia, in Culture Of Animal Cells (R. I. Freshney, Alan R. Liss, Inc., 1987).

A further embodiment relates to a polypeptide comprising at least one sequence as defined in SEQ ID NO: 2 to 17, 19 to 32, 34, 35, 37 and/or 38 and/or a fragment and/or derivative thereof. The polypeptide can be made available by expression of a polynucleotide or by chemical synthesis.

The amino acid sequences of the present invention also encompass all sequences that differ from the sequences disclosed herein as a result of amino acid insertions, deletions and substitutions. Amino acid “substitutions” are preferably the result of replacing an amino acid by another amino acid having similar structural and/or chemical properties, that is to say conservative amino acid exchanges. Amino acid substitutions may be made on the basis of a similarity in polarity, charge, solubility, hydrophobicity, hydrophilicity and/or the amphiphatic nature of the residues included. For example, non-polar (hydrophobic) amino acids include alanine, leucine, isoleucine, valine, proline, phenylalanine, tryptophan and methionine; polar neutral amino acids include glycine, serine, threonine, cysteine, tyrosine, asparagine and glutamine; positively charged (basic) amino acids include arginine, lysine and histidine; and negatively charged (acidic) amino acids include aspartic acid and glutamic acid.

“Insertions” or “deletions” typically occur in the range of 1-3 amino acids. The allowed variation can be determined by experiment, by systematically making insertions, deletions or substitutions of amino acids in a polypeptide molecule using DNA recombination techniques and testing the resulting recombinant variants with respect to their activity, for which the person skilled in the art is not required to go beyond the performance of routine experiments.

For example, the polypeptide can also be in the form of a chimeric polypeptide encoded by a fusion gene, which comprises at least one further sequence. This additional sequence can serve the purpose of, for example, facilitating purification of the expression product or providing the expression product with an additional function.

Examples of additional sequences facilitating purification are so-called tags, which will be known to the person skilled in the art, for example the his-tag.

In addition, the present invention relates to use of at least one sequence as defined in SEQ ID NO: 1, 18, 33 and/or 36 and/or at least one fragment thereof and/or at least one polypeptide as defined in SEQ ID NO: 2 to 17, 19 to 32, 34, 35, 37 and/or 38 and/or at least one fragment thereof in the production of a pharmaceutical composition for the treatment of undesirable cell growth or undesirable-cell proliferation in an individual. The composition may comprise, for example, a suitable vector together with auxiliary factors which make possible the expression of a tubulysin, preferably in the undesirable cells, and as a result prevent further growth or further proliferation of those cells. The composition may also comprise cells according to the invention which have been transfected with a vector, for example a tubulysin-expressing vector.

In a preferred embodiment, the undesirable cell growth or undesirable cell proliferation is a tumour. The tumour may be a benign growth or a malignant growth.

In a further embodiment, the undesirable cell growth is a pathogenic infection, in which case the pathogen may be single-celled or multi-celled. This also includes infections with fungi, for example Candida or Aspergillus, and infections with parasites, for example trypanosomes or schistosomes. In a preferred embodying form of use, the pathogenic infection is a mycosis, malaria or a parasitic disease.

The invention relates furthermore to a pharmaceutical composition comprising at least one polynucleotide as defined in SEQ ID NO: 1, 18, 33 and/or 36 and/or at least one fragment thereof and/or at least one polypeptide as defined in SEQ ID NO: 2 to 17, 19 to 32, 34, 35, 37 and/or 38 and/or at least one fragment thereof. The compositions comprise a therapeutically active amount or dose of the active ingredient or component in question. A therapeutically active dose relates to that amount of the compound which is sufficient to produce an alleviation of symptoms, for example treatment, cure, prevention or alleviation of such conditions, especially inhibition or prevention of undesirable cell growth and cell proliferation, in a patient. Suitable administration routes include, for example, parenteral administration, including intramuscular and subcutaneous injections and also intrathecal, direct intraventricular, intravenous and intraperitoneal injections.

In a further embodiment, the pharmaceutical composition comprises at least one pharmaceutically acceptable carrier. Such a composition may further comprise (in addition to the component and carrier) diluents, fillers, salts, buffers, stabilisers, solubility enhancers and other materials well known in the prior art. The expression “pharmaceutically acceptable” means a non-toxic material which does not impair the efficacy of the biological activity of the active component(s). The properties of the carrier depend on the administration route. The therapeutic composition may furthermore comprise further agents or active substances which improve the activity or efficacy or facilitate use during treatment. Such additional factors and/or agents may be included in the therapeutic composition in order to produce a synergistic effect or to minimise side-effects. Techniques for formulation, preparation and administration of the compounds of the present invention are to be found in “Remington's Pharmaceutical Sciences”, Mack Publishing Co., Easton, Pa., latest edition.

In addition, the present invention relates to a method of producing tubulysins and tubulysin biosynthesis proteins, comprising the steps:
(a) expression of at least one polynucleotide as defined in SEQ ID NO: 1, 18, 33 and/or 36 and/or at least one fragment thereof and/or at least one polypeptide as defined in SEQ ID NO: 2 to 17, 19 to 32, 34, 35, 37 and/or 38 and/or at least one fragment thereof, and
(b) purification of the expression products.
   Methods for the expression of proteins are known to the person skilled in the art and can be found from the relevant literature, for example from Methods In Enzymology, Vols. 154 and 155 (Wu et al. eds.) or Recombinant DNA Methodology (R. Wu ed., Academic Press). For the purification of expression proteins a large number of methods are known to the person skilled in the art. In addition to chromatographic methods such as, for example, affinity chromatography or HPLC, immunological procedures such as, for example, immobilised antibodies against an epitope on the expression product, for example a His-tag, can also be used for purification of the products.

In a preferred embodiment, expression is carried out in prokaryotic or eukaryotic cells and/or by in vitro expression. The expression of polypeptides in prokaryotic or eukaryotic cells is a frequently used method and is generally achieved by means of an expression vector as described hereinbefore. Vectors have likewise already been described for in vitro expression. These, and the necessary factors, are commercially available in the form of kits, for example from BioRad, Stratagene, Invitrogen and Clontech.

The invention relates moreover to a method of finding genes which are involved in the biosynthesis of tubulysins. The method comprises the following steps:
(a) hybridisation of at least one polynucleotide as defined in SEQ ID NO: 1, 18, 33 and/or 36 and/or at least one fragment thereof with DNA, RNA and/or cDNA of a species that is not identical to Angiococcus disciformis, and
(b) isolation and characterisation of the hybridised DNA, RNA and/or cDNA.
   The hybridisation can be carried out under conditions of differing stringency.

The stringency of the hybridisation, as used herein, relates to conditions under which polynucleotide double strands are stable. As the person skilled in the art will know, the stability of a double strand is a function of the sodium ion concentration and temperature (see, for example, Sambrook et al., Molecular Cloning: A Laboratory Manual 2^{nd} Ed. (Cold Spring Harbor Laboratory, (1989)). The levels of stringency used for the hybridisation can be readily adapted by the person skilled in the art.

The expression “low-stringency hybridisation” denotes conditions which are equivalent to hybridisation in 10% formamide, 5× Denhart's solution, 6×SSPE, 0.2% SDS at 42° C., followed by washing in 1×SSPE, 0.2% SDS at 50° C. Denhart's solution and SSPE, like other suitable hybridisation buffers, are well known to the person skilled in the art.

“Moderate-stringency hybridisation” means conditions which allow DNA to bind to a complementary nucleic acid that has approximately 60% identity, preferably approximately 75% identity, especially approximately 85% identity, with that DNA, special preference being given to identity of more than approximately 90% with that DNA. Moderate-stringency conditions are preferably conditions which are equivalent to hybridisation in 50% formamide, 5× Denhart's solution, 5×SSPE, 0.2% SDS at 42° C., followed by washing in 0.2×SSPE, 0.2% SDS at 65° C.

High-stringency hybridisation means conditions which allow hybridisation only of those nucleic acid sequences which form stable double strands in 0.018M NaCl at 65° C. (i.e., when a double strand is not stable in 0.018M NaCl at 65° C., it is not stable under the high-stringency conditions described/defined herein).

Nucleic acid hybridisation techniques can be used, moreover, in order to identify and obtain a nucleic acid which is encompassed by the present invention. In brief, any nucleic acid having a certain homology to a sequence disclosed in this invention or a fragment thereof can be used as a probe for identification of a similar nucleic acid by hybridisation under moderate-stringency to high-stringency conditions. Such similar nucleic acids can then be isolated, sequenced and analysed in order to determine whether they are encompassed by the present invention.

In addition, the present invention makes available a kit for the production of tubulysins, comprising:
(a) at least one polynucleotide comprising a sequence as defined in SEQ ID NO: 1, 18, 33 or 36 or a fragment thereof and/or at least one vector comprising such a polynucleotide or
(b) suitable media and buffers for the multiplication of cells which allow expression of the polynucleotide and/or vector, and
(c) suitable means for purification of the expression product(s).

By virtue of their action on the tubulin skeleton and their cytotoxicity, especially in the case of fungi, tubulins are also suitable as a disinfectant which can reduce or prevent contamination with tubulin-containing cells.

The invention accordingly relates also to use of a composition comprising at least one polypeptide as defined in SEQ ID NO: 2 to 17, 19 to 32, 34, 35, 37 and/or 38 and/or at least one biologically active fragment or derivative thereof as a disinfectant. In addition to the polypeptide defined above, other substances having a disinfecting action can also be present in the disinfectant provided that they do not inhibit the action of the polypeptide according to the invention. In addition, the disinfectant can comprise further adjuvants such as, for example, buffers, water, dyes, fragrances, stabilisers, carriers etc.

In a preferred embodiment, the composition is liquid or in powder form.

Accordingly, the invention relates also to disinfectants as defined above.

Insofar as no other definitions are given, all technical and scientific expressions used herein have the same meaning as that usually understood by the skilled person in the field to which the invention is directed. All publications, patent applications, patents and other references mentioned herein are included in their entirety by way of reference. However, in the event of a conflict, the present description, including the definitions, shall be decisive. In addition, the materials, methods and examples are merely illustrative and should not be interpreted as being limiting.

1. Identification of the Tubulysin Biosynthesis Cluster in Angiococcus disciformis An d48 as a Result of Mariner Transposon Mutagenesis Using pMycoMar

Identification of the tubulysin biosynthesis cluster was carried out by constructing a transposon mutant bank from Angiococcus disciformis An d48 using pMycoMar.

Rubin & Mekalanos (Proc. Natl. Acad. Sci. USA, 96 (1999), 1645-1650) developed, from the mariner element Himar1, the plasmid pMycoMar, which constitutes a simple transposition system capable of efficiently infecting bacteria in vivo and generating insertion mutants. This plasmid comprises the mini-transposon magellan4, in which the Tn5 kanamycin resistance gene and oriR6K are flanked by the inverted repeats of Himar1. In addition, Himar1 transposase was cloned into the mycobacterial temperature-sensitive replicon pPR23 under the transcriptional control of the T6 promoter. pMycoMar likewise encodes a gentamycin resistance gene.

On transposition, Himar1 is distinguished by a TA dinucleotide recognition sequence. It can therefore randomly integrate into a host genome and, statistically speaking, switch off all active genes by means of an insertion mutation. On the basis of that fact, the intention was to generate a mutant bank from An d48 and identify the tubulysin biosynthesis cluster by means of a knockout mutant.

Alternatively, it is also possible to start from Archangium gephyra DSM 11092 and to proceed in accordance with a protocol of Biozym Diagnostic (Oldendorf, DE; catalogue TSM99K2; pEZ::TN<KAN-2> Tnp transposome kit).

1.1 Generation of the Mutant Bank

Two different protocols were used for electrotransformation of A. disciformis An d48. These protocols were established for the myxobacteria Stigmatella aurantiaca (Stamm & Plaga, Arch. Microbiol., 172 (1995), 483-494) and Myxococcus xanthus (Kashefi & Hartzell, Mol. Microbiology, 15 (1995) 483-494). The two methods showed no difference in the transformation efficiency of A. disciformis An d48 so that the electrotransformation for construction of the transposon bank was carried out according to the protocol for Stigmatella aurantiaca. The two protocols are described hereinbelow.

1.1.1. Electrotransformation of Angiococcus disciformis An d48 According to the Stigmatella aurantiaca Protocol

An A. disciformis culture grown in 50 ml of tryptone medium (10 g of tryptone; 2 g of MgSO4₂; 0.1% vitamin B12 [10 ng/ml]; 0.2% glucose per 1 litre of medium; pH 7.2) is cultured at 30° C. to 2*10⁸ cells/ml. On the basis of a generation time of 6 hours, this culture was inoculated the day before so that, as calculated, this cell density would be achieved. The culture is centrifuged at 20° C. (20 min; 4000 rpm) and the cells are resuspended in the same volume of washing buffer (5 mM HEPES/NaOH, 0.5 mM CaCl₂; pH 7.2). After centrifuging again, they are resuspended in 25 ml of buffer and centrifuged again. Before that centrifugation step, the absolute cell count in the 25 ml is determined so that, as calculated, 1*10⁹ cells/40 μl are resuspended.

Electroporation Conditions:

1-3 μg of DNA and 40 μl of cell suspension are mixed and transferred into an electroporation cuvette (0.1 cm) cooled on ice. The electroporation is performed at 200Ω, 25 mF and 0.85 kV/cm.

Immediately after the electroporation, 1 ml of tryptone medium is added. After transfer into 50 ml of tryptone medium, the cells are shaken for 6 h at 30° C. to allow phenotypic expression. The culture is then centrifuged (20 min, 4000 rpm, 20° C.) and resuspended in 1 ml of tryptone. On the basis of a 100% survival rate for the cells, a dilution series is produced and 1*10⁸-1*10⁴ cells are plated with 3 ml of tryptone soft agar onto kanamycin-containing (50 μg/ml) tryptone plates. The plates are incubated at 30° C. and the first clones can be seen after 5-8 days.

1.1.2. Electrotransformation of Angiococcus disciformis An d48 According to the Myxococcus xanthus Protocol

The growth conditions of the preculture and main culture and the centrifugations and subsequent concentration of the cell count were exactly as described under 1.1.1. This was optimised in a manner that departs from the standard protocol for Myxococcus xanthus.

Electroporation Conditions:

1-3 μg of DNA and 40 μl of cell suspension are mixed and transferred into an electroporation cuvette (0.1 cm) cooled on ice. The electroporation is performed at 400Ω, 25 μF and 0.65 kV/cm.

Immediately after the electroporation, 1 ml of tryptone medium is added and shaking is carried out in a 1.5 ml Eppendorf reagent vessel for 6 h at 30° C. On the basis of a 100% survival rate for the cells, a dilution series is produced and 1*10⁸-1*10⁴ cells are plated with 3 ml tryptone soft agar onto kanamycin-containing (50 μg/ml) tryptone plates. The plates are incubated at 30° C. and after 5-8 days the first clones/mutants can be seen, which were picked using an inoculation loop.

1.2 Culturing of Transposon Mutants

The mutants were incubated in 96-well microtitre plates in 200 μl of M7 medium (5 g of Probion; 1 g of CaCl₂; 1 g of MgSO4₄; 1 g of yeast extract; 5 g of starch; 10 g of HEPES; 0.1% vitamin B12 [10 ng/ml] per 1 litre of medium; pH 7.4) at 32° C., and after 10 days a copy of the entire bank was produced. For the purpose, 50 μl of culture of each mutant were transferred with 100 μl of M7 medium to new microtitre plates. After incubation for a further seven days, a copy was frozen at −80° C. to provide long-term cultures. The remaining copy of the bank was extracted and the extract was tested for generated tubulysin knockout mutants by means of a toxicity test.

When mutants were identified which exhibited changes with respect to the wild type in this analysis (no cell nucleus fragmentation), these were recultured from the long-term culture. For control of the results obtained, 50 ml of M7-medium large cultures of the mutants in question had to be tested again. In the case of possible tubulysin knockout mutants, the extracts were first fractionated by means of an HPLC run and the fractions were then tested for tubulysin by means of a toxicity test. The prior fractionation avoided masking of the tubulysin action by myxothiazole. Because the two secondary metabolites have different retention times on elution from a C-14 column, they are each contained in different fractions in the following toxicity test.

1.3 Toxicity Test

After culturing, the mini-cultures from the 96-well microtitre plates were concentrated to dryness by nitrogen-blowing on a heating block at 37° C. Afterwards, the cell pellets were resuspended in 100 μl of methanol over 2 h, and 10 μl were used in each case for the following toxicity test in order to be able to detect tubulysin production by the mutant in question.

For this test, L929 cells are cultured in DMEM medium (Invitrogen, Groningen) at 37° C. and then carefully harvested using a cell scraper. This cell suspension is then diluted 1:10 with DMEM, and 120 μl are distributed per hole of a 96-well microtitre plate. 10 μl of cell extract of the individual transposon mutants are then added thereto and incubated for five days at 37° C. After that incubation period, the L929 cells are examined under a microscope for cell nucleus fragmentation, which is a sign of tubulysin action. In the case of cells that did not exhibit cell nucleus fragmentation, the mutants in question were identified as presumable tubulysin knockout mutants. The extracts of those mutants were grown in 50 ml of M7 medium (+1 ml of XAD-16 absorber resin from Rohm & Haas) and the cell nuclei of the L929 cells were, after completion of a toxicity test, additionally tested for cell nucleus fragmentation or tubulysin production by staining of the chromosome by means of DAPI staining.

1.4 Determination of the Integration Gene Site of Tubulysin Knockout Mutants in the An d48 Mariner Mediated Mutant Bank by Means of Transposon Recovery

In the generated mutant bank it was possible to identify, by means of the toxicity test, five mutants (MutT176, 524, 781, 794 and 929) which produced no tubulysin. It was possible to confirm that result after reculturing the mutants from the long-term culture and re-analysis. In order to obtain information as to the region of the genome in which the Himar1 element is transposed, a transposon recovery was carried out. In this method, the chromosomal DNA of the mutant in question is cut using different restriction enzymes which do not cut within the known magellan4 sequence. The restricted DNA is ligated and, after transformation into DH5α/λpir cells, incubation on kanamycin-containing LB plates is carried out at 37° C. On those plates only those E. coli cells can grow which comprise a plasmid with magellan4 and consequently the Tn5 kanamycin resistance gene. At the ends of the transposon, such a plasmid comprises chromosomal DNA from An d48. These plasmids can replicate in the E. coli cells DH5α/λpir because oriR6K is located within the transposon sequence. The transposon was accordingly isolated from the genome in question and sequenced with the primers K388 and K389. The sequences obtained were then tested against the gene bank for homologies with known genes and, in the process, showed high degrees of similarity with non-ribosomal peptide synthetases (NRPS) from known secondary metabolite biosynthesis gene clusters such as those of myxothiazole, nostopeptolide and saframycin. These analyses gave clear indications that the sequences were sequence fragments from the sought tubulysin gene cluster. By means of restriction analyses and Southern analyses, the size of the individual transposon plasmids and their relative integration sites with respect to one another (within the gene cluster) were determined.

1.4.1. Transposon Recovery

Isolation of chromosomal DNA according to standard protocols from 50 ml of tryptone medium culture of each A. disciformis An d48 mutant. 5 μg of this DNA are used for the following cloning-out of the transposon, with a restriction first being carried out. In the process, the enzymes NotI and BamHI were used, which have no restriction site within magellan4 and statistically should cut relatively frequently in GC-rich DNA.

Digestion of genomic DNA with NotI and BamHI:

| | |
|---|---|
| | 5 μg of DNA |
| + | 3 μl of 10x NEB buffer |
| + | 3 μl of 100x BSA |
| + | 10 U of restriction enzyme (BamHI or NotI) |
| + | x μl of dist. H₂O |
| | 30 μl batch incubated for 3 h at 37° C. [symbol] again 10 U of |
| | enzyme added to the restriction batches and incubated for a |
| | further 2 h at 37° C. |

Precipitation of the Restricted DNA and Subsequent Ligation

1 vol. of chloroform/phenol is added to the entire restriction batch and centrifugation is carried out for 10 min. (13,200 rpm; 20° C.). The supernatant is transferred to a new reaction vessel and 1/10 vol. of 3M NaOAc and 2.5 vol. of 100% EtOH are added. For precipitation of the DNA, the reaction vessel is incubated for 1 h at −20° C. and is then centrifuged for 30 min. (13,200 rpm; 4° C.). The supernatant is discarded and the pellet is washed three times with 70% EtOH, centrifuging each time for 5 min. (13,200 rpm; 20° C.). After discarding the supernatant, the pellet is dried at 37° C. and resuspended in 15 μl of H₂O. For the subsequent ligation, the entire 15 μl of precipitated DNA are used.

Ligation batch:

| | |
|---|---|
| | 15 μl of DNA |
| + | 4 μl of 5x ligase buffer (NEB) |
| + | 1 μl of NEB ligase |
| | 20 μl batch incubated overnight at 16° C. [symbol] 1 μl |
| | of ligase again added to the ligation batches and |
| | incubated overnight at 16° C. |

Electrotransformation of the Ligation Batches into the E. coli Strain DH5α-λpir

1-3 μl of the ligation batches and 50 μl of DH5α-λpir cells are mixed and transferred into an electroporation cuvette (0.1 cm) cooled on ice. The electroporation is performed at 200Ω, 25 mF and 1.25 kV/cm. The cells are then suspended in 1 ml of LB medium (10 g of tryptone; 10 g of NaCl; 5 g of yeast extract per 1 litre of medium) and incubated for 1 h at 37° C. They are then plated onto kanamycin-containing (50 μg/ml) LB plates. After incubating for one day at 37° C., the clones can be picked. Only those cells can grow which have a transposon plasmid and accordingly a Tn5-Kan^{R}-mediated resistance.

1.5 Sequence Evaluation of the Tubulysin Biosynthesis Gene Cluster from pMutT794/NotI

The transposon plasmid pMutT794/NotI comprises 52985 bp chromosomal DNA from Angiococcus disciformis An d48. Together with the Himar1 mini-transposon magellan4 (2199 bp), which is integrated into the plasmid at base pair 37317 bp, 55184 bp were sequenced. In total, 21760 bp originate from coding genes of the tubulysin gene cluster and 31219 bp from further coding genes. These ORFs are, in some cases, regulator genes which can influence the expression of tubulysin. Sequence comparisons with the transposon plasmids of the other tubulysin knockout mutants showed that magellan4 in the case of the mutants MutT781 (36975 bp) and MutT929 (36197 bp) is transposed into the biosynthesis gene cluster within 1658 bp of MutT794.

In the sequence, the start of the tubulysin gene cluster includes three NRPS modules (tubA-C), a cyclodeaminase-encoding gene (tubZ) and a PKS module (tubD). Also located within the gene cluster are an anion transporter-encoding gene (ORF1), which serves for transporting the tubulysin out of the cell, and a further ORF (ORF2). The basic arrangement of the genes, and of the individual domains with an N-methyltransferase within the adenylation domains (A) of tubB and tubC, corresponds to the typical structure of the gene cluster and the tubulysin biosynthesis associated therewith. However, in contrast to the known gene cluster structures, the methyltransferase domains (NMT) are not located between the adenylation and thiolation domains (PCP) but rather between A8 and A9 within the adenylation domain (A) (highly conserved regions within the adenylation domains of NRPS; Konz & Marahiel, Chem. Biology, 6 (1999) R39-R48). TubA encodes an incomplete condensation domain, which is theoretically not required for biosynthesis. The polyketide synthase (PKS) located at the end of the known sequence comprises a ketoacyl synthase (KS) acyl transferase (AT) and ketoreductase (KR) domain.

The remaining sequence of the tubulysin biosynthesis gene cluster was identified from a cosmid bank of An d48 (held at the DSMZ) under standard conditions. The PKS module (tubD) ending the first half of the sequence is continued by the afore-mentioned KS, AT and KR domains and furthermore comprises an enoyl reductase (ER) and an acyl carrier protein (ACP). In the following sequence of tubD, an NRPS is encoded which carries a heterocyclisation (HC), adenylation (A) and peptidyl carrier protein (PCP) domain. The genes tubE and tubF also follow. The gene tubE encodes an NRPS with the domains C, A and PCP. On tubF, a PKS having the following domain arrangement is encoded: ketoacyl synthase (KS), acyl transferase (AT), ketoreductase (KR), C-methyltransferase (CMT), dehydratase (DH), enoyl reductase (ER), acyl carrier protein (ACP) and finally a thioesterase which serves for removal of the finished tubulysin in the form of a free acid from the multienzyme complex. The insertion site of the transposon magellan4 is located in the case of MutT176 at base pair 54579 within the biosynthesis gene cluster. The insertion site of the mutant MutT524 is not located on the gene cluster sequence known to us. We therefore postulate that the insertion site is located within an acyl transferase-encoding gene which is located downstream from the tubulysin biosynthesis gene cluster and has a post-translational function for the modification of tubulysin.

2. Identification of the Connection Sequence of the Tubulysin Biosynthesis Gene Cluster from Angiococcus disciformis An d48

2.1 Identification and Characterisation of Cosmids which Carry an Overlapping Sequence Downstream from the Tubulysin Biosynthesis Gene Cluster

The previous example described how the first half of the tubulysin biosynthesis gene cluster, together with further genes involved in biosynthesis, was identified and annotated by means of mariner-based transposon mutagenesis and subsequent transposon recovery. Because genes encoding both monooxygenases and also acyl transferases are absent within that sequence, a further sequence downstream therefrom had also to be identified and characterised. The afore-mentioned genes should be encoded within that sequence because they are necessary for biosynthesis of tubulysin. The biosynthesis gene cluster should, as a result, be identified in its entirety.

For the purpose, a cosmid bank was produced from A. disciformis An d48 by means of a Gigapack II XL packaging kit (from Stratagene) in E. coli SURE. Within that bank, cosmids having a relatively long overlap with the tubulysin biosynthesis gene cluster downstream from tubF should be identified. For the purpose, two primer pairs were derived from the known sequence of the tubulysin gene cluster and the PCR amplification products were used as probes for the following hybridisation of the cosmid bank. The first primer pair ASTls1A-1B yields a 889 bp DNA fragment and is located 1 kb upstream from the NotI restriction cutting site in tubD. The second primer pair ASTls2A-2B generates a 700 bp fragment, which is located in tubC 11 kb away upstream in the known cluster end. The PCR was carried out at an annealing temperature of 54° C. As a result of that hybridisation it was possible to identify various cosmids. By means of PCR and restriction analysis, they were examined with respect to the size of their overlap with the known cluster sequence. For the purpose, the primer pairs ASTls1A/B and ASTls2A/B were again used at an annealing temperature of 58° C. In the case of restriction, various enzymes were used in single and double restrictions.

Because, after the restriction analyses, the cosmids F7 and F13 exhibited a similarly large overlap with the first portion of the cluster, one of these cosmids carries the genetic information necessary to identify the genes directly associated with the cluster.

2.1.1 Southern Analysis of the Cosmids F7 and F13

For identification of the correct cosmid, restriction enzymes were initially selected which cut as infrequently as possible and at the end of the known gene cluster sequence. The enzymes selected were NdeI and NsiI, which cut at the positions 39306 bp and 39430 bp, respectively. Furthermore, both enzymes cut only once more in the known sequence. Using a generated probe (primer pair Tls_{up/down}), which binds behind those cuts directly at the end of the known cluster sequence, the cosmid gene bank should then be “screened”. For the purpose, the cosmids were hydrolysed in various double restriction batches and separated on an agarose gel (0.8%). For the double restriction, the enzymes BamHI, EcoRI and NotI were selected in addition to NdeI and NsiI. The combinations with EcoRI and NotI were intended to result in a fragment being identified, by means of the hybridisation, which extends to the end of the cosmid insert in question. If that fragment should be too large for subsequent cloning, BamHI was also used in order to obtain, where appropriate, shorter fragments. The hybridisation was carried out at 42° C. and washing was carried out under high-stringency conditions (68° C.).

The result of this analysis was that a 12 kb fragment was detected in the EcoRI/NdeI restriction batch of the F7 cosmid. This fragment comprises the remaining sequence of the tubulysin gene cluster and extends to the end of the insert sequence of the cosmid. This conclusion was drawn from the restriction analyses and the characterised overlap with the tubulysin gene cluster sequence. The detected NotI/NdeI fragment resulted in a size of 4.2 kb. Therefore, at least one further NotI cutting site must be located within the 12 kb insert sequence of F7—between the start (NdeI—restriction site) and vector (scos). Consequently, the connection sequence can be cloned and sequenced in smaller fragments (as NdeI/NotI and NotI/NotI fragments). The BamHI/NsiI double restriction batch yielded five fragments in total.

2.1.2. Cloning of the Rest of the Tubulysin Gene Cluster Sequence from Cosmid F7

The cosmid F7 was cut in a double restriction batch using the restriction endonucleases NsiI and EcoRI (2 h; 37° C.). After separation of the restriction batch using 0.8% agarose gel, the corresponding band was cut out of the gel and extracted with the NucleoSpin kit (from Macherei-Nagel). The isolated fragment was re-cut using NotI in order to check whether the hybridisation results achieved were confirmed. In addition, it was checked whether further NotI recognitions sequences are located within the 12 kb connection sequence in order to be able to determine the number of partial fragments to be cloned. After separation on an 0.8% agarose gel, the restriction produced a 4.2 kb fragment (NsiI/NotI fragment) and a 8 kb fragment (NotI/NotI fragment).

Firstly, the hybridisation result could be confirmed and both fragments could be used for cloning. Secondly, it was confirmed that the fragment is the correct fragment, which carries the sequence downstream from the cluster.

The 12.2 kb NsiI/EcoRI fragment and also the 4.2 kb (NsiI/NotI) and 8 kb (NotI/NotI) fragments were cloned into the vector pUC18. The vector was cut using PstI/EcoRI or PstI NotI and NotI for the following ligation. PstI and NsiI have a compatible cutting pattern so that, after successful cloning, those cutting sites are no longer present. Using HindIII or NdeI and EcoRI, the 12 kb insert can be cut out again from the pUC18-derivative using a double restriction.

The clones obtained were checked with respect to their correctness by means of those restriction batches. One of those clones (ASpUC12) was used for a following in vitro transposition by means of the GPS™-1 Genome Priming System (from New England Biolabs_{Inc}).

2.1.3. In Vitro Transposition Using GPS-1™ Genome Priming System

Using the GPS-1 system, the intention was to sequence the cloned NsiI/NotI fragment by means of an in vitro trans-position based on Tn7. This “kit” uses a TnsABC transposase, which randomly inserts the transposon (Transprimer™) into the target sequence. By means of specific sequencing primers (PrimerN/PrimerS), which can be “read out” at the flanking ends of the transposon, the adjacent regions of the DNA insert can be sequenced. Because the transposon is randomly inserted into the target sequence, the entire target sequence can be characterised by sequencing a certain number of generated transposon mutants.

Procedure for in vitro transposition

| | |
|---|---|
| | 2 μl of 10x GPS buffer |
| + | 1 μl of pGPS 1.1 (provides Kan^{R}) |
| + | 0.2 μl of target DNA (corresponds to 80 ng of ASexp7) |
| + | 14.8 μl of dH₂O |
| | 18 μl batch |

The batch is mixed well and 1 μl of TnsABC transposase is added (again mixing well). The entire reaction batch is incubated for 10 min. at 37° C. so that the transposase mixes in the reaction batch before the actual reaction. After the addition of 1 μl of “start solution”, the reaction batch is incubated for one hour at 37° C. During that period, the strand transfer of the transposon into the target DNA occurs. The reaction is then terminated by incubation for 10 min. at 75° C. From that batch, 2 μl were transformed into E. coli DH10B and plated onto kanamycin-containing medium. A total of about 2000 clones have grown after incubation overnight at 37° C.

20 of those clones were examined with regard to the ratio in which the transposon has been inserted into the insert or vector. For the purpose, those clones were hydrolysed in a double restriction batch using the endonucleases EcoRI and HindIII. The restriction analysis showed that, in the case of 75% of the clones, the transposon had been inserted into the insert. Consequently, 192 clones were sequenced, as a result of which an approximately 12-fold coverage of the sequence was achieved (in the case of a read length of 500 bp per sequencing).

2.2 Sequence Analysis and Annotation of the 12 kb Connection Sequence

The remaining sequence obtained for the tubulysin biosynthesis gene cluster is 12,219 bp long and has an overlap with the previously identified sequence of 133 bp. Sequence portions which had been covered only once were subjected to double-strand-sequencing by repeated sequencing of specific clones. In this sequence, an acyl transferase is encoded by base pair 6416-6898 (position 76,787-77,545 bp in the overall sequence). The other identified ORFs likewise have a function in tubulysin biosynthesis. The entire sequence is accordingly 82,868 bp.

3. Identification of a Tubulysin-Overproducing Mutant within the Mariner Transposon Mutant Bank

In order to investigate the mutants of the transposon bank with regard to further noteworthy phenotypes compared to the wild type, an HPLC analysis was carried out. In the process it was checked whether insertion of the transposon into chromosomal regions of biosynthesis gene clusters of other expressed secondary substances had occurred. In those comparisons with respect to an extract of the wild type, non-producing mutants of the metabolites in question should then be identified. Those metabolites include myxothiazole (Gerth et al. 1980 J Antibiot (Tokyo) 33(12):1474-1479 and Silakowski et al. 1999 J Biol. Chem. 274(52):37391-9, myxochelin (Gerth et al. 1983 J Antibiot (Tokyo) 36(9):1150-6. and Silakowski et al. 2000 Eur J. Biochem. 267(21):6476-85) and angiolam (Kunze et al. 1985, J Antibiot (Tokyo) 38(12):1649-54).

In evaluations of the 1,200 HPLC runs, extracts of a number of mutants were noted in which increased myxothiazole production could be measured. In order to check the results obtained, 50 ml M7 medium cultures of the mutants in question were tested again and time kinetics were produced for myxothiazole production over several days compared to the wild type. The results of those tests showed clearly increased production of myxothiazole in the various mutants compared to the An d48 wild type.

Determination of the transposon insertion site within the mutant in question was carried out by means of “transposon recovery” and subsequent sequencing of the flanking regions (see 1.4). The sequences obtained were investigated for homologies with known genes and showed high degrees of similarity with regard to regulatory elements/genes from bacterial organisms. On the basis of those results, the entire mutant bank was investigated for tubulysin-overproducing mutants. For the purpose, the existing toxicity test (see 1.3) was optimised. As a result of multiple dilutions of the respective mutant extract used in the toxicity test, a dilution of the tubulysin is achieved and consequently the characteristic action on L929 cells is no longer detectable from a certain dilution. Using those dilution series (from the entire mutant bank), mutants were identified where significantly higher dilutions are required in order not to be able to detect any action. This means that the mutant in question exhibits increased tubulysin production.

It was possible to identify mutant Mut158, which exhibited a four-fold increase in tubulysin D production. This result was shown both by culture of the mutant in 50 ml cultures by way of HPLC-MS tests, and also a number of kinetics with a subsequent optimised toxicity test against the wild type. By means of the toxicity test, even eight-fold overproduction of tubulysins was established, in which case the overall action of all tubulysin derivatives was detected and not only that of tubulysin D. Mutant 158 consequently exhibits, entirely surprisingly, overexpression of further tubulysins compared to the wild type of A. disciformis. On no account was this to be expected. Cloning-out of the genomic region directly at the insertion site of the transposon and sequencing were carried out as described under 1.4.

The sequence of the gene concerned shows high degrees of similarity with a protein kinase (from Stigmatella aurantiaca), the insertion site of the transposon constituting the promoter region of this gene. Without being bound to this mechanism of action, this gene has a negative regulatory function for tubulysin formation, which is why inactivation of the gene results in increased production. The entire sequence comprises 2,200 bp, the protein kinase being encoded by base pair 1,228-20 and having a total size of 1,209 bp. The ORF located upstream encodes a tubulysin biosynthesis protein and has a size of 933 bp.

Primer Sequences:

Sequencing primer for the Himar1 mini-transposon magellan4

SEQ ID NO: 39

| | | |
|---|---|---|
| K-388: | 5′→3′ | ^{5′}TGG GAA TCA TTT GAA GGT TGG^{3′} |

SEQ ID NO: 40

| | | |
|---|---|---|
| K-389: | 3′→5′ | ^{5′}TGT GTT TTT CTT TGT TAG ACC G^{3′} |

Primer pair ASTls1A/B was derived from tubD and produces an 889 bp fragment

| | |
|---|---|
| AST1s1A | |
| ^{5′}CAC CCG GAC CTG CCT GGA TTC^{3′} | SEQ ID NO: 41 |
| AST1s1B | |
| ^{5′}TGC TCG GCT GGC GCT ACT CAC^{3′} | SEQ ID NO: 42 |

Primer pair ASTls2A/B was derived from tubC and produces a 700 bp fragment

| | |
|---|---|
| AST1s2A | |
| ^{5′}GCT CCC GGG CCA CGT GGT TGA AGA^{3′} | SEQ ID NO: 43 |
| AST1s2B | |
| ^{5′}CCG CGG GCC GTG GCA GTG GTG TA^{3′} | SEQ ID NO: 44 |

Primer pair Tlsᵤₚ/Tls_{down} was derived from tubF and produces a 125 bp fragment

| | | |
|---|---|---|
| T1sᵤₚ | ^{5′}TGG CAG CCA GCC CGA GC^{3′} | SEQ ID NO: 45 |
| T1s_{down} | ^{5′}CCG CGG GTG CCC TCT CAT C^{3′} | SEQ ID NO: 46 |

| Name of gene (whether | | | | | Classification of domain | |
|---|---|---|---|---|---|---|
| PKS or NRPS) | | | | | and position in | |
| Function | Coding region | Coding region on the | Size in bp | | nucleotide sequence or | |
| SEQ ID NO of amino acid | in sequence | minus strand | Size of derived/translated | | protein of greatest | |
| sequence | (SEQ ID NO:) | (SEQ ID NO:) | protein in Da | | similarity | |
| ORF16: | 3,308-1 | 79,561-82,866 | 3,308 | bp | 49% identity, 64% | |
| Valyl-tRNA-synthase | (SEQ ID NO: 1) | (SEQ ID NO: 18) | 122,434 | Da | similarity to Valyl-tRNA | |
| SEQ ID NO: 32 | | | | | synthetase [Thermotoga | |
| | | | | | maritima] | |
| | | | | | NP_229614 | |
| ORF15: | 4,706-3,453 | 78,163-79,416 | 1,254 | bp | 38% identity, 61% | |
| Regulatory component of a | (SEQ ID NO: 1) | (SEQ ID NO: 18) | 41,337 | Da | similarity to “response” | |
| sensory transduction system | | | | | regulator CheY sub- | |
| SEQ ID NO: 31 | | | | | family [Synechocystis sp.] | |
| | | | | | NP_440346 | |
| ORF14: | 5,719-7,164 | | 1,446 | bp | 29% identity, 44% | |
| Two-component “response” | (SEQ ID NO: 1) | | 52,449 | Da | similarity to “response” | |
| regulator | | | | | regulator protein | |
| SEQ ID NO: 2 | | | | | [Mesorhizobium loti] | |
| | | | | | NP_102571 | |
| ORF13: | 9,557-7,317 | 73,312-75,552 | 2,241 | bp | 29% identity, 46% | |
| Two-component regulation | (SEQ ID NO: 1) | (SEQ ID NO: 18) | 83,814 | Da | similarity to two- | |
| system comprising sensor | | | | | component regulation | |
| kinase/“response” regulator | | | | | system of sensor kinase/ | |
| hybrid | | | | | “response” regulator | |
| SEQ ID NO: 30 | | | | | hybrid [Agrobacterium | |
| | | | | | tumefaciens] | |
| | | | | | NP_535879 | |
| ORF12: | 12,193-10,550 | 70,676-72,319 | 1,644 | bp | 70% identity, 85% similarity | |
| Heat shock protein | (SEQ ID NO: 1) | (SEQ ID NO: 18) | 58,650 | Da | to heat shock protein GroEL | |
| SEQ ID NO: 29 | | | | | [Rhodothermus marinus] | |
| | | | | | AAD37976 | |
| ORF11: | 12,841-13,881 | | 1,041 | bp | 34% identity, 56% similarity | |
| Tubulysin biosynthesis | (SEQ ID NO: 1) | | 37,818 | Da | to hypothetical protein | |
| protein | | | | | [Corynebacterium | |
| SEQ ID NO: 3 | | | | | glutamicum] | |
| | | | | | NP_616546 | |
| ORF10: | 14,833-13,835 | 68,036-69,034 | 999 | bp | 33% identity, 47% similarity | |
| Tubulysin biosynthesis | (SEQ ID NO: 1) | (SEQ ID NO: 18) | 36,441 | Da | to conserved hypothetical | |
| protein | | | | | protein [Streptomyces | |
| SEQ ID NO: 28 | | | | | coelicolor] | |
| | | | | | NP_631315 | |
| ORF9: | 14,942-15,586 | | 645 | bp | 30% identity, 51% similarity | |
| Transcription regulator | (SEQ ID NO: 1) | | 23,789 | Da | to transcription regulator, | |
| SEQ ID NO: 4 | | | | | TetR family [Caulobacter | |
| | | | | | crescentus] | |
| | | | | | NP_420005 | |
| ORF8: | 15,847-16,983 | | 1137 | bp | 24% identity, 40% similarity | |
| Integrase | (SEQ ID NO: 1) | | 41,614 | Da | to integrase | |
| SEQ ID NO: 5 | | | | | [Corynebacterium | |
| | | | | | glutamicum] | |
| | | | | | NP_601233 | |
| ORF7: | 21,154-18,809 | 61,715-64,016 | 2,346 | bp | 33% identity, 47% similarity | |
| Tubulysin biosynthesis | (SEQ ID NO: 1) | (SEQ ID NO: 18) | 89,255 | Da | to hypothetical protein | |
| protein | | | | | [Nostoc sp.] | |
| SEQ ID NO: 27 | | | | | NP_490333 | |
| ORF6: | 22,366-23,532 | | 1,167 | bp | 32% identity, 46% similarity | |
| Serine/threonine-kinase | (SEQ ID NO: 1) | | 43,228 | Da | to serine/threonine-kinase | |
| SEQ ID NO: 6 | | | | | Pkn14 [Myxococcus xanthus] | |
| | | | | | AAK64427 | |
| ORF5: | 24,591-26,513 | | 1,923 | bp | 42% identity, 56% similarity | |
| Protein kinase | (SEQ ID NO: 1) | | 68,825 | Da | to protein kinase [Stigmatella | |
| SEQ ID NO: 7 | | | | | aurantiaca] | |
| | | | | | CAD19078 | |
| ORF4: | 26,597-27,517 | | 921 | bp | 33% identity, 37% similarity | |
| Adenine deaminase | (SEQ ID NO: 1) | | 33,507 | Da | to adenine deaminase-related | |
| SEQ ID NO: 8 | | | | | protein [Deinococcus | |
| | | | | | radiodurans] | |
| | | | | | NP_285591 | |
| ORF3: | 29,858-30,400 | | 543 | bp | 24% identity, 41% similarity | |
| Cytosine deaminase | (SEQ ID NO: 1) | | 20,880 | Da | to possible cytosine | |
| SEQ ID NO: 9 | | | | | deaminase [Salmonella | |
| | | | | | typhimurium] | |
| | | | | | NP_462244 | |
| tubA | 31,220-32,392 | | 1,173 | bp | similarity to C-domain core | |
| SEQ ID NO: 10 | (SEQ ID NO: 1) | | 43,202 | Da | motifs C2-C3 | |
| ORF2 | 33,056-32,397 | 49,813-50,472 | 660 | bp | 29% identity, 42% similarity | |
| Tubulysin biosynthesis | (SEQ ID NO: 1) | (SEQ ID NO: 18) | 25,390 | Da | to conserved hypothetical | |
| protein | | | | | protein [Neurospora crassa] | |
| SEQ ID NO: 26 | | | | | CAD11370 | |
| tubZ | 34,195-33,074 | 48,674-49,795 | 1,122 | bp | 39% identity, 53% similarity | |
| Lysine cyclodeaminase | (SEQ ID NO: 1) | (SEQ ID NO: 18) | 40,499 | Da | to lysine cyclodeaminase | |
| “pipicolinic acid | | | | | [Streptomyces hygroscopicus] | |
| synthase” | | | | | CAA60467 | |
| SEQ ID NO: 25 | | | | | | |
| ORF1 | 35,422-34,205 | 47,447-48,664 | 1,218 | bp | 40% identity, 65% similarity | |
| ATP-dependent anion | (SEQ ID NO: 1) | (SEQ ID NO: 18) | 46,030 | Da | to anion-transporting ATPase | |
| transporter | | | | | [Aquifex aeolicus] | |
| SEQ ID NO: 24 | | | | | NP_213468 | |
| tubB | 35,522-40,147 | | 4,626 | bp | C 35,747-36,769 | 76-416 |
| SEQ ID NO: 11 | (SEQ ID NO: 1) | | 170,704 | Da | A 37,184-39,817 | 555-1,432 |
| | | | | | NMT 38,369-39,730 in | 950-1,403 |
| | | | | | A domain between core motif | |
| | | | | | A8 and A9 | |
| | | | | | PCP 39,818-40,069 | 1,433-1,516 |
| tubC | 40,144-48,021 | | 7,878 | bp | C 40,372-41,397 | 77-418 |
| SEQ ID NO: 34 | (SEQ ID NO: 1) | | 289,141 | Da | A 41,824-43,215 | 561-1,024 |
| | 1-7,878 | | | | PCP 43,216-43,461 | 1,025-1,106 |
| | (SEQ ID NO: 33) | | | | C 43,552-44,574 | 1,137-1,477 |
| | | | | | A 44,980-47,631 | 1,613-2,496 |
| | | | | | NMT 46,153-47,547 in A | 2,004-2,468 |
| | | | | | domain between core motif | |
| | | | | | A8 and A9 | |
| | | | | | PCP 47,632-47,868 | 2,497-2,575 |
| tubD | 48,011-58,558 | | 10,548 | bp | KS 48,011-49,321 | 1-437 |
| SEQ ID NO: 12 | (SEQ ID NO: 1) | | 383,778 | Da | AT 49,622-50,584 | 538-858 |
| | | | | | KR 51,473-52,309 | 1,155-1,433 |
| | | | | | ER 53,066-53,980 | 1,686-1,990 |
| | | | | | ACP 54,158-54,460 | 2,050-2,150 |
| | | | | | HC 54,461-55,870 | 2,151-2,620 |
| | | | | | A 56,000-57,412 | 2,664-3,134 |
| | | | | | PCP 57,413-57,643 | 3,135-3,211 |
| tubE | 58,551-62,096 | | 3,546 | bp | C 58,689-59,714 | 47-388 |
| SEQ ID NO: 35 | (SEQ ID NO: 1) | | 130,337 | Da | A 60,156-61,697 | 536-1,049 |
| | 18.408-21.953 | | | | PCP 61,698-61,967 | 1,050-1,139 |
| | (SEQ ID NO: 33) | | | | | |
| tubF | 62,103-70,616 | | 8,514 | bp | KS 62,127-63,320 | 9-406 |
| SEQ ID NO: 13 | (SEQ ID NO: 1) | | 309,369 | Da | AT 63,711-64,676 | 537-858 |
| | | | | | KR 64,959-65,882 | 953-1,260 |
| | | | | | CMT 65,985-67,061 | 1,295-1,653 |
| | | | | | DH 67,242-67,829 | 1,714-1,909 |
| | | | | | ER 68,247-69,128 | 2,049-2,342 |
| | | | | | PCP 69,360-69,605 | 2,420-2,501 |
| | | | | | TE 69,759-70,586 | 2,553-2,828 |
| ORF17: | 71,640-70,583 | 11,229-12,284 | 1,056 | bp | 24% identity, 40% similarity | |
| similarity to patatin- | (SEQ ID NO: 1) | (SEQ ID NO: 18) | 38,371 | Da | to patatin-similar protein | |
| similar protein (lipid | | | | | [Anabaena sp. 90] | |
| acylhydrolase) | | | | | CAC01602 | |
| SEQ ID NO: 23 | | | | | | |
| ORF18: | 72,786-71,731 | 10,083-11,138 | 1,056 | bp | 24% identity, 40% similarity | |
| similarity to patatin- | (SEQ ID NO: 1) | (SEQ ID NO: 18) | 38,371 | Da | to patatin-similar protein | |
| similar protein (lipid | | | | | [Anabaena sp.90] | |
| acylhydrolase) | | | | | CAC01602 | |
| SEQ ID NO: 22 | | | | | | |
| ORF19: | 75,209-74,655 | 7,660-8,214 | 555 | bp | 31% identity, 43% similarity | |
| Tubulysin biosynthesis | (SEQ ID NO: 1) | (SEQ ID NO: 18) | 20,040 | Da | to hypothetical protein | |
| protein | | | | | [Azotobacter vinelandii] | |
| SEQ ID NO: 21 | | | | | ZP_00092207 | |
| ORF20: | 75,488-76,645 | | 1,158 | bp | 41% identity, 62% similarity | |
| Tubulysin biosynthesis | (SEQ ID NO: 1) | | 43,282 | Da | to hypothetical protein | |
| protein | | | | | [Microbulbifer degradans] | |
| SEQ ID NO: 14 | | | | | ZP_00065421 | |
| tubG: | 76,787-77,545 | | 759 | bp | 47% identity, 53% similarity | |
| Acyltransferase | (SEQ ID NO: 1) | | 28,039 | Da | to N-hydroxyarylamine O- | |
| SEQ ID NO: 15 | | | | | acetyltransferase | |
| | | | | | [Streptomyces avermitilis] | |
| | | | | | NP_826733 | |
| ORF21: | 77,769-78,695 | | 927 | bp | 28% identity, 39% similarity | |
| Tubulysin biosynthesis | (SEQ ID NO: 1) | | 33,859 | Da | to conserved hypothetical | |
| protein | | | | | protein [Xanthomonas | |
| SEQ ID NO: 16 | | | | | axonopodis] | |
| | | | | | NP_641500 | |
| ORF22: | 79,138-80,019 | | 882 | bp | 37% identity, 49% similarity | |
| Tubulysin biosynthesis | (SEQ ID NO: 1) | | 32,668 | Da | to hypothetical protein | |
| protein | | | | | [Rhizobium etli] | |
| SEQ ID NO: 17 | | | | | NP_659913 | |
| ORF23: | 81,319-80,057 | 1,550-2,812 | 1,263 | bp | 34% identity, 52% similarity | |
| Tubulysin biosynthesis | (SEQ ID NO: 1) | (SEQ ID NO: 18) | 49,133 | bp | to hypothetical protein | |
| protein | | | | | [Nostoc punctiforme] | |
| SEQ ID NO: 20 | | | | | ZP_00109292 | |
| ORF24: | 82,797-81,721 | 72-1,148 | 1,077 | bp | 31% identity, 44% similarity | |
| Carboxylate reductase | (SEQ ID NO: 1) | (SEQ ID NO: 18) | 37,621 | Da | to pyrroline-carboxylate | |
| SEQ ID NO: 19 | | | | | reductase NosF | |
| | | | | | [Nostoc sp.] | |
| | | | | | AAF17284 | |
| ORF25: | 1,228-20 | 973-2181 | 1,209 | bp | Tubulysin-overproducing | |
| Protein kinase | | (SEQ ID NO: 36) | 44,079 | Da | mutant | |
| SEQ ID NO: 38 | | | | | | |
| ORF26: | 2,157-1,225 | 44-976 | 933 | bp | Tubulysin-overproducing | |
| Tubulysin biosynthesis | | (SEQ ID NO: 36) | 33,229 | Da | mutant | |
| protein | | | | | | |
| SEQ ID NO: 37 | | | | | | |

KS: ketoacyl synthase
AT: acyl transferase
KR: ketoreductase
DH: dehydratase
ER: enoyl reductase
ACP: acyl carrier protein
CMT: C-methyltransferase
NMT: N-methyltransferase
A: adenylation domain
C: condensation domain
PCP: peptidyl carrier protein
TE: thioesterase
bp: base pairs
Da: dalton

1. An ssDNA molecule selected from the following group:
(i) an ssDNA molecule having a sequence according to FIG. 1;
(ii) an ssDNA molecule which is 90, 91, 92, 93, 94, 95, 96, 97, 98, 99, or 100% homologous to an ssDNA molecule according to (i) in respect of its number of nucleotides or its nucleotide sequence but which differs by at least one nucleotide from the ssDNA molecule according to (i) in respect of its number of nucleotides and/or its nucleotide sequence; and
(iii) an ssDNA molecule having a sequence which is complementary to the sequence of an ssDNA molecule according to (i) or (ii).

2. A dsDNA molecule comprising an ssDNA molecule according to claim 1 and a strand complemetary thereto.

3. An ssDNA molecule selected from the following group:
(i) am ssDNA molecule having a sequence of positions 3.308 to 1 (ORF 16) of the sequence according to FIG. 1;
(ii) an ssDNA molecule having a sequence of positions 4706 to 3453 (ORF 15) of the sequence according to FIG. 1;
(iii) an ssDNA molecule having a sequence of positions 5719 to 7164 (ORF 14) of the sequence according to FIG. 1;
(iv) an ssDNA molecule having a sequence of positions 9557 to 7317 (ORF 13) of the sequence according to FIG. 1;
(v) an ssDNA molecule having a sequence of positions 12193 to 10550 (ORF 12) of the sequence according to FIG. 1;
(vi) an ssDNA molecule having a sequence of positions 12841 to 13881 (ORF 11) of the sequence according to FIG. 1;
(vii) an ssDNA molecule having a sequence of positions 14833 to 13835 (ORF 10) of the sequence according to FIG. 1;
(viii) an ssDNA molecule having a sequence of positions 14942 to 15586 (ORF 9) of the sequence according to FIG. 1;
(ix) an ssDNA molecule having a sequence of positions 15847 to 16983 (ORF 8) of the sequence according to FIG. 1;
(x) an ssDNA molecule having a sequence of positions 21154 to 18809 (ORF 7) of the sequence according to FIG. 1;
(xi) an ssDNA molecule having a sequence of positions 22366 to 23532 (ORF 6) of the sequence according to FIG. 1;
(xii) an ssDNA molecule having a sequence of positions 24591 to 26513 (ORF 5) of the sequence according to FIG. 1;
(xiii) an ssDNA molecule having a sequence of positions 26597 to 27517 (ORF 4) of the sequence according to FIG. 1;
(xiv) an ssDNA molecule having a sequence of positions 29858 to 30400 (ORF 3) of the sequence according to FIG. 1;
(xv) an ssDNA molecule having a sequence of positions 31220 to 32392 (TubA) of the sequence according to FIG. 1;
(xvi) an ssDNA molecule having a sequence of positions 33056 to 32397 (ORF 2) of the sequence according to FIG. 1;
(xvii) an ssDNA molecule having a sequence of positions 34195 to 33074 (TubZ) of the sequence according to FIG. 1;
(xviii) an ssDNA molecule having a sequence of positions 35422 to 34205 (ORF 1) of the sequence according to FIG. 1;
(xix) an ssDNA molecule having a sequence of positions 35522 to 40147 (TubB) of the sequence according to FIG. 1;
(xx) an ssDNA molecule having a sequence of positions 40144 to 48021 (TubC) of the sequence according to FIG. 1;
(xxi) an ssDNA molecule having a sequence of positions 48011 to 58558 (TubD) of the sequence according to FIG. 1;
(xxii) an ssDNA molecule having a sequence of positions 58551 to 62096 (TubE) of the sequence according to FIG. 1;
(xxiii) an ssDNA molecule having a sequence of positions 62103 to 70616 (TubF) of the sequence according to FIG. 1;
(xxiv) an ssDNA molecule which is hybridisable with a molecule according to (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii), or (xxiii) under stringent conditions and especially has the same number of bases; and;
(xxv) an ssDNA molecule which is 90, 91, 92, 93, 94, 95, 96, 97, 98, 99, or 100% homologous to an ssDNA molecule according to (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii), or (xxiii) in respect of its number of nucleotides or its nucleotide sequence but which differs by at least on nucleotide from that ssDNA molecule in respect of its number of nucleotides and/or its nucleotide sequence; and
(xxvi) an ssDNA molecule having a sequence which is complementary to the sequence of a molecule according to (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii), (xxiii), (xxiv) or (xxv).

4. A dsDNA molecule comprising and ssDNA molecule according to claim 3 and a strand complemtary thereto.

5. An ssDNA molecule selected from the following group:
(i) an ssDNA molecule having a sequence of positions 35747 to 36769 (domain C of the tubB gene) of the sequence according to FIG. 1;
(ii) an ssDNA molecule having a sequence of positions 37184 to 39817 (domain A of the tubB gene) of the sequence according to FIG. 1;
(iii) an ssDNA molecule having a sequence of positions 38369 to 39730 (domain NMT of the tubB gene) of the sequence according to FIG. 1;
(iv) an ssDNA molecule having a sequence of positions 39818 to 40069 (domain PCP of the tubB gene) of the sequence according to FIG. 1;
(v) an ssDNA molecule having a sequence of positions 40372 to 41397 (domain C of the tubC gene) of the sequence according to FIG. 1;
(vi) an ssDNA molecule having a sequence of positions 41824 to 43215 (domain PCP of the tubC gene) of the sequence according to FIG. 1;
(vii) an ssDNA molecule having a sequence of positions 43216 to 43461 (domain PCP of the tubC gene) of the sequence according to FIG. 1;
(viii) an ssDNA molecule having a sequence of positions 43552 to 44574 (domain C of the tubC gene) of the sequence according to FIG. 1;
(ix) an ssDNA molecule having a sequence of positions 44980 to 47631 (domain A of the tubC gene) of the sequence according to FIG. 1;
(x) an ssDNA molecule having a sequence of positions 46153 to 47547 (domain NMT of the tubC gene) of the sequence according to FIG. 1;
(xi) an ssDNA molecule having a sequence of positions 47632 to 47868 (domain PCP of the tubC gene) of the sequence according to FIG. 1;
(xii) an ssDNA molecule having a sequence of positions 48011 to 49321 (domain KS of the tubD gene) of the sequence according to FIG. 1;
(xiii) an ssDNA molecule having a sequence of positions 49622 to 50584 (domain AT of the tubD gene) of the sequence according to FIG. 1;
(xiv) an ssDNA molecule having a sequence of positions 51473 to 52309 (domain KR of the tubD gene) of the sequence according to FIG. 1;
(xv) an ssDNA molecule having a sequence of positions 53066 to 53980 (domain ER of the tubD gene) of the sequence according to FIG. 1;
(xvi) an ssDNA molecule having a sequence of positions 54158 to 54460 (domain ACP of the tubD gene) of the sequence according to FIG. 1;
(xvii) an ssDNA molecule having a sequence of positions 54461 to 55870 (domain HC of the tubD gene) of the sequence according to FIG. 1;
(xviii) an ssDNA molecule having a sequence of positions 56000 to 57412 (domain A of the tubD gene) of the sequence according to FIG. 1;
(xix) an ssDNA molecule having a sequence of positions 57413 to 57643 (domain PCP of the tubD gene) of the sequence according to FIG. 1;
(xx) an ssDNA molecule having a sequence of positions 58689 to 59714 (domain C of the tubE gene) of the sequence according to FIG. 1;
(xxi) an ssDNA molecule having a sequence of positions 60156 to 61697 (domain A of the tubE gene) of the sequence according to FIG. 1;
(xxii) an ssDNA molecule having a sequence of positions 61698 to 61967 (domain PCP of the tubE gene) of the sequence according to FIG. 1;
(xxiii) an ssDNA molecule having a sequence of positions 62127 to 63320 (domain KS of the tubF gene) of the sequence according to FIG. 1;
(xxiv) an ssDNA molecule having a sequence of positions 63711 to 64676 (domain AT of the tubF gene) of the sequence according to FIG. 1;
(xxv) an ssDNA molecule having a sequence of positions 64959 to 65882 (domain KR of the tubF gene) of the sequence according to FIG. 1;
(xxvi) an ssDNA molecule having a sequence of positions 65985 to 67061 (domain CMT of the tubF gene) of the sequence according to FIG. 1;
(xxvii) an ssDNA molecule having a sequence of positions 67242 to 67829 (domain DH of the tubF gene) of the sequence according to FIG. 1;
(xxviii) an ssDNA molecule having a sequence of positions 68247 to 69128 (domain ER of the tubF gene) of the sequence according to FIG. 1;
(xxix) an ssDNA molecule having a sequence of positions 69360 to 69605 (domain PCP of the tubF gene) of the sequence according to FIG. 1;
(xxx) an ssDNA molecule having a sequence of positions 69759 to 70586 (domain TE of the tubF gene) of the sequence according to FIG. 1;
(xxxi) an ssDNA molecule which is hybridisable with a molecule according to (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii), (xxiii), (xxiv), (xxv), (xxvi), (xxvii), (xxviii), (xxix) or (xxx) under stringent conditions and especially has the same number of bases;
(xxxii) an ssDNA molecule which is 90, 91, 92, 93, 94, 95, 96, 97, 98, 99, or 100% homologous to an ssDNA molecule according to (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii), (xxiii), (xxiv), (xxv), (xxvi), (xxvii), (xxviii), (xxix) or (xxx) in respect of its number of nucleotides or its nucleotide sequence but which differs by at least one nucleotide from that ssDNA molecule in respect of its number of nucleotides and/or its nucleotide sequence; and
(xxxiii) an ssDNA molecule having a sequence which is complementary to the sequence of a molecule according to (i), (ii), (iii), (iv), (v), (vi), (vii), (viii), (ix), (x), (xi), (xii), (xiii), (xiv), (xv), (xvi), (xvii), (xviii), (xix), (xx), (xxi), (xxii), (xxiii), (xxiv), (xxv), (xxvi), (xxvii), (xxviii), (xxix), (xxx), (xxxi) or (xxxii).

6. A dsDNA molecule comprising an ssDNA molecule according to claim 5 and a strand complementary thereto.

7. Variants or mutants which result from a substitution, insertion or deletion of nucleotides or from an inversion of nucleotide segments of an ssDNA molecule according to claim 1, those variants and mutants encoding enzyme variants or enzyme mutants for the production of secondary substance(s) having properties characteristic of tubulysins.

8. RNA
(a) having a sequence corresponding to that of an ssDNA molecule according to claim 1 or
(b) having a sequence of an RNA according to (a) but in the opposite direction (anti-sense), or
(c) having a sequence of an RNA according to (a) or (b) and having a strand complementary thereto, in each case optionally as an element of a recombinant vector.

9. A vector, especially an expression vector, having a DNA molecule according to claim 1.

10. A cell, especially for expression, into which a DNA molecule or a vector according to claim 8 has been integrated.

11. A cell according to claim 10, the cell being derived from culturable bacteria, especially Myxobacteria, preferably Angiococcus, especially A. disciformis, Archangium, especially A. gephyra, Escherichia coli, pseudomonads or actinomycetes.

12. Use of a vector according to claim 8 for the transformation of cells of organisms for the transient or permanent expression of one or more proteins (expression product(s) which is/are encoded by a DNA (ssDNA or dsDNA) of the vector).

13. Use of a cell according to claim 10 for the enzymatic biosynthesis, metasynthesis or partial synthesis of a tubulysin, especially tubulysin A, B, C, D, E and/or F.

14. An expression product of a DNA molecule according to claim 1.

15. A polynucleotide comprising a sequence as defined in SEQ ID NO: 1, 18, 33 or 36, or a fragment thereof.

16. A polynucleotide according to claim 15, wherein the fragment is a CDS defined in the sequence protocol.

17. A vector comprising a polynucleotide according to claim 15.

18. A cell comprising a vector according to claim 17.

19. A polypeptide comprising at least one sequence as defined in SEQ ID NO: 2 to 17, 19 to 32, 34, 35, 37 and/or 38 and/or a fragment and/or derivative thereof.

20. Use of at least one polynucleotide as defined in SEQ ID NO: 1, 18, 33 and/or 36 and/or at least one fragment thereof and/or at least one polypeptide as defined in SEQ ID NO: 2 to 17, 19 to 32, 34, 35, 37 and/or 38 and/or at lease one fragment thereof in the production of a pharmaceutical composition for the treatment of undesirable cell growth or undesirable cell proliferation in an individual.

21. Use according to claim 20, wherein the undesirable cell growth or undesirable cell proliferation is a tumour.

22. Use according to claim 20, wherein the undesirable cell growth or undesirable cell growth is a pathogenic infection.

23. Use according to claim 22, wherein the pathogenic infection is a mycosis, malaria, or a parasitic disease.

24. A pharmaceutical composition comprising at least one polynucleotide as defined in SEQ ID NO: 1, 18, 33 and/or 36 and/or at least one fragment thereof and/or at least one polypeptide as defined in SEQ ID NO: 2 to 17, 19 to 32, 34, 35, 37 and/or 38 and/or at least one fragment thereof.

25. A pharmaceutical composition according to claim 24, which further comprises at least one pharmaceutically acceptable carrier.

26. A method of producing tubulysins and tubulysin biosynthesis proteins, comprising the steps:
(a) expression of at least one polynucleotide as defined in SEQ ID NO: 1, 18, 33 and/or 36 and/or at least one fragment thereof and/or at least one polypeptide as defined in SEQ ID NO: 2 to 17, 19 to 32, 34, 35, 37 and/or 38 and/or at least one fragment thereof, and
(b) purification of the expression products.

27. A method according to claim 26, wherein expression is carried out in prokaryotic or eukaryotic cells and/or by in vitro expression.

28. A method of finding genes which are involved in the biosynthesis of tubulysins, comprising the steps:
(a) hybridization of at least one polynucleotide as defined in SEQ ID NO: 1, 18, 33 and/or 36 and/or at least one fragment therof with DNA, RNA and/or cDNA of a species that is not identical to Angiococcus disciformis, and
(b) isolation and characterization of the hybridized DNA, RNA and/or cDNA.

29. A kit for the production of tubulysins, comprising:
(a) at least one polynucleotide according to claim 15 or
(b) suitable media and buffers for the multiplication of cells which allow expression of the polynucleotide and/or vector and
(c) suitable means for purification of the expression product(s).

30. Use of a composition comprising at least one polypeptide as defined in SEQ ID NO: 2 to 17, 19 to 32, 34, 35, 37 and/or 38 and/or at least one biologically active fragment or derivative thereof as a disinfectant.

31. Use according to claim 30, wherein the composition is liquid or in powder form.

32. A disinfectant as defined in claim 30.

33. Variants or mutants which result from a substitution, insertion or deletion of nucleotides or from an inversion of nucleotide segments of a dsDNA molecule according to claim 2, those variants and mutants encoding enzyme variants or enzyme mutants for the production of secondary substance(s) having properties characteristic of tubulysins.

34. A kit for the production of tubulysins, comprising:
(a) at least one vector according to claim 17 or
(b) suitable media and buffers for the multiplication of cells which allow expression of the polynucleotide and/or vector and
(c) suitable means for purification of the expression product(s).
